(19) United States
(12) Patent Application Publication
(10) Pub. No.: US 2014/0272767 A1
(43) Pub. Date: Sep. 18, 2014

(54) SYSTEM AND METHOD FOR OPTICAL IMAGING, MAGNIFICATION, FLUORESCENCE, AND REFLECTANCE

(71) Applicant: Convergent Dental, Inc., Natick, MA (US)

(72) Inventor: Nathan P. Monty, Shrewsbury, MA (US)

(21) Appl. No.: 14/212,432

(22) Filed: Mar. 14, 2014

Related U.S. Application Data
(60) Provisional application No. 61/793,059, filed on Mar. 15, 2013.

(51) Int. Cl. A61N 5/06; A61B 5/00

(52) U.S. Cl. CPC A61N 5/0613; A61B 5/0088
USPC 433/27; 433/29

(57) ABSTRACT

A laser-based dental treatment system also provides for imaging of the treatment area during treatment thereof, without requiring the operator to switch between different devices. A laser beam delivery subsystem and an imaging system are coupled such that at least a portion of the path along which the light reflected from the treatment area propagates towards the imaging system is substantially the same as at least a portion of the path along which the laser beam propagates. Optionally, an illumination system that can direct light to a candidate treatment area for diagnosis thereof, and/or to provide adequate light to the treatment area for imaging, is also be integrated with the treatment system.

### CROSS-REFERENCE TO RELATED APPLICATIONS

This application claims benefit of priority to U.S. Provisional Patent Application No. 61/793,059, entitled “System and Method for Optical Imaging, Magnification, Fluorescence, and Reflectance,” filed on Mar. 15, 2013, the disclosure of which is incorporated herein by reference in its entirety.

### FIELD OF THE INVENTION

The present invention generally relates to imaging and diagnosis and, in particular, to integrated intra-oral imaging and treatment.

### BACKGROUND

For years, dental operators have used hand-held dental mirrors that can be inserted in the mouth of a patient for reflecting images of regions inside the mouth, so that the dental operator can view the area to be treated. This technique has several disadvantages. First, it is often difficult to hold the dental mirror in an appropriate position in order to reflect images of an area to be treated. Second, it can be difficult to ensure that adequate light is directed to the treatment area within the mouth for reflection of the light by the dental mirror, so that the operator can inspect the area to be treated. Another disadvantage is that the images seen in a dental mirror cannot be readily shared with other people, e.g., the patient, colleagues, dental assistants, or students, for example, to discuss the treatment.

To alleviate some of these problems, intra-oral cameras are commonly used to obtain photographs of the teeth and/or other regions of the mouth. Any required or recommended treatment can be conveyed or explained to the patient and/or others using such photographs. Optical microscopy may also be used, e.g., to zoom in and focus on the treatment area, to aid in diagnosis and/or inspection thereof. Different optical imaging techniques, including optical fluorescence and optical reflectance, may also be used for the detection and identification of affected carious tissue.

Electronic video endoscopes typically use a miniature camera, e.g., a charge coupled device (CCD) and an optical fiber, to capture and transport images to a monitor. Such video endoscopes are generally available in various sizes, but they are typically rather small and tubular so that they can be inserted into a body cavity or surgical opening. Some endoscopes include a light source located at an end to provide adequate light to illuminate the area to be imaged. Typical video endoscopes are not specifically designed for dental applications and, hence, are generally not suitable for such applications. For example, it is very difficult, if not impossible, to view the lingual aspects of the teeth using video endoscopes, due to their tubular shape.

Various intraoral camera devices are merely imaging devices and, as such, they must be used interchangeably with a dental treatment device such as a conventional burr or a laser device. Dentists preforming a treatment procedure using laser-based devices typically rely on viewing the treatment area directly or via a conventional hand-held mirror. Direct viewing is often awkward and does not provide the dentist with an adequate visual acuity or sufficient clarity to accurately and efficiently perform the procedure. The use of viewing tools, such as a standard dental mirror or even an intra-oral camera, is often impractical because the dentist must frequently switch between the treatment device and the viewing device. In addition to inconvenience to the patient and the operator, the frequent switching can be potentially harmful with a laser device, as the laser beam may be inadvertently directed to parts of the patient's body that are not to be treated or to other persons. The danger of unwanted exposure to a laser beam also exists if a dental mirror is used during treatment, as the mirror can reflect the laser beam.

The health of teeth, e.g., the presence of caries, plaque, or bacterial infection of teeth, can be determined by visual inspection or by using X-rays. With a visual inspection, satisfactory results often cannot be achieved because it is difficult to inspect and diagnose certain tooth regions, as described above. Although X-rays can be very effective in ascertaining caries and other tooth diseases, due to their potentially harmful effects, X-rays are typically not preferred during early stages of diagnosis.

Some contactless diagnosis devices for the determination of caries, plaque, and/or bacterial infection in teeth, irradiate a tooth with a virtually monochromatic light source. In response to such irradiation, a fluorescence radiation is excited by the bacteria located in decayed tissue of the tooth. The fluorescence spectrum can manifest clear differences between healthy and affected regions of the tooth. Thus, a healthy portion of the tooth or tissue can be distinguished from an affected portion of the tooth or tissue. A difference in the light that is reflected in response to irradiation of the tooth with a monochromatic light source can also be used to distinguish healthy tissue from affected tissue. A significantly high water content of a typical affected tissue relative to the water content in enamel, for example, can cause a change in the reflection of light from such affected tissue relative to the reflection off enamel or other unaffected tissue.

Similar to the viewing/imaging devices, the diagnostic devices are also not suitable for treatment and, hence, must be used iteratively with a treatment device. As frequent interchanging of devices can increase patient discomfort and may cause safety concerns, as described above, dental operators usually inspect a treatment area before starting the treatment, but minimize or avoid inspections using viewing and/or diagnostic devices during treatment. This can significantly limit the ability of a dental operator to assess the effect of partially completed treatment. It can also prevent or delay the discovery of additional areas of a tooth or tissue that may need treatment because, in some instances, these additional areas become visible only after a part of the treatment procedure has been completed. The ability to frequently assess the effect of treatment is important in laser-based treatment, so as to minimize unintended and undesired treatment or ablation. Systems and methods are therefore needed to provide a dental operator with improved visual acuity, sufficient clarity, and an appropriate field of view while performing laser-based treatment, in a convenient and safe manner.

### SUMMARY OF THE INVENTION

In various embodiments of a laser-based treatment and imaging system one or more components of the imaging subsystem are coupled to and/or integrated with one or more components of the treatment subsystem in such manner that assessment of a dental area during treatment thereof is facilitated. This is achieved, in part, by configuring the imaging subsystem such that at least a portion of the path along which light reflected from the treatment area, representing images thereof, propagates is substantially the same as at least a portion of the path along which the laser beam for treatment propagates. Therefore, imaging can be performed nearly simultaneously with treatment, without having to swap in and out different devices for treatment and imaging.

Some embodiments also include an illumination subsystem. The light from a source mounted on the operator's head or on an articulating arm can be obstructed by the treatment and/or imaging device. Light from the illumination system integrated with the treatment and imaging system, however, is not significantly obstructed by the components of that system. As such, the illumination system can enhance the quality of imaging. Additionally or in the alternative, the illumination subsystem can include a predominantly monochromatic, or narrow-spectrum source of light, e.g., blue or red light, that can be directed to a candidate area to be treated or an area that has been at least partially treated, so as to detect any affected portions of such areas.

Accordingly, in one aspect an apparatus for imaging a dental treatment area includes an optical subsystem and an imaging system. The galvo-controlled optical subsystem is adapted for directing a laser beam from a laser source to a dental treatment area. The laser beam is directed along an optical axis. The imaging system is positioned at a location different than a location of the laser source, and includes (i) a viewer, and (ii) an imaging optical subsystem. The imaging optical subsystem is adapted to receive light rays from the treatment area, after the light rays travel substantially along the optical axis and via the galvo-controlled optical subsystem, for delivery to the viewer.

The imaging optical subsystem may include an adjustable focus lens mechanism to adjust focus of an image received in the viewer and/or to magnify the images. The adjustable focus lens mechanism may include a motorized lens stack and/or a liquid lens. The viewer may include a camera. In various embodiments, the galvo-controlled optical subsystem includes a first galvo-controlled mirror and a second galvo-controlled mirror. The imaging optical subsystem may be adapted to receive light rays reflected from any one of the first and second galvo-controlled mirrors. The first galvo-controlled mirror may include an optically transmissive mirror, and the imaging optical subsystem may be adapted to receive light rays passing through the optically transmissive mirror.

In some embodiments the apparatus includes an illumination system for directing light to the treatment area. The illumination system may include a first light source that includes one or more light sources of substantially monochromatic light, such as, a light emitting diode (LED), a laser diode (LD), etc. The substantially monochromatic light may have a peak wavelength range of either about 600-700 nm or about 375-475 nm. The first light source may include an optically transmissive element, such as a Fresnel lens.

In some embodiments, the first light source is located such that light therefrom is directed to the treatment area via the galvo-controlled optical subsystem. The galvo-controlled optical subsystem may include a galvo-controlled mirror, and the first light source may be located such that light therefrom reflects off the galvo-controlled mirror. The galvo-controlled optical subsystem may also include an optically transmissive element, and the first light source can be located such that light therefrom passes through the optically transmissive element. In some embodiments, the first light source is located such that light therefrom is directed to the treatment area independently of the galvo-controlled optical subsystem. The illumination system may also include a second light source, e.g., of white light, broad-spectrum light, and/or narrow-spectrum or substantially monochromatic light.

In another aspect, an apparatus for imaging a dental treatment area includes a first beam splitter and a galvo-controlled optical subsystem for directing a laser beam from a laser source to a dental treatment area. The laser beam is directed via the first beam splitter, and along an optical axis. The apparatus also includes an imaging system positioned at a location different than a location of the laser source. The imaging system includes (i) a viewer, and (ii) an imaging optical subsystem. The imaging optical subsystem is adapted to receive light rays from the treatment area, after the light rays travel substantially along the optical axis and via the first beam splitter, for delivery to the viewer. The apparatus also includes an illumination system for directing light to the treatment area. The illumination system includes a first light source, e.g., of substantially monochromatic light or white light.

The imaging optical subsystem may include an adjustable focus lens mechanism to adjust focus of an image received in the viewer. The adjustable focus lens mechanism may include one or more of a motorized lens stack and a liquid lens. In some embodiments, the viewer includes a camera. The galvo-controlled optical subsystem may include a first galvo-controlled mirror and a second galvo-controlled mirror. The first galvo-controlled mirror may include an optically transmissive mirror.

In some embodiments, the first light source is a source of substantially monochromatic light, and may include a light emitting diode (LED), a laser diode (LD), or both. The substantially monochromatic light may have a peak wavelength range of about 600-700 nm or about 375-475 nm. The first light source may also include an optically transmissive element, e.g., a Fresnel lens. The first light source may be located such that light therefrom is directed to the treatment area independently of the galvo-controlled optical subsystem. The illumination system may include a second light source, e.g., a source of white light, broad-spectrum light, and/or narrow spectrum or substantially monochromatic light.

In some embodiments, the apparatus includes a second beam splitter. The laser beam may be directed to the dental treatment area independently of the second beam splitter. The imaging optical subsystem may be located such that the light rays received thereby travel via the second splitter. The illumination system is located such that the light therefrom travels via the second splitter, as well.

In another aspect, a method of identifying a dental area for laser treatment includes illuminating a candidate area for dental treatment using a source of substantially monochromatic light. The light is passed via a hand piece adapted for passing therethrough and along an optical axis thereof a laser beam for treatment. The method also includes generating, at an imaging system, an image formed by light rays reflected from the candidate treatment area and traveling along the optical axis of the hand piece. Furthermore, the method includes identifying within the generated image a region corresponding to received light rays having a wavelength different than a peak wavelength of the substantially monochromatic source of light, and designating the identified region as the dental area for laser treatment. In some embodiments, the method further includes directing a laser beam to the designated dental area via the hand piece. The laser beam may be (i) generated by a source positioned at a location different than a location of the imaging system, and (ii) may travel substantially along the optical axis of the hand piece.

In another aspect, a method of treating a dental area using a laser includes directing a laser beam to a designated dental area via a galvo-controlled optical subsystem and via a hand piece, along an optical axis thereof. The method also includes generating, at an imaging system, an image formed by light rays reflected from the designated dental area, after the reflected light rays travel along the optical axis of the hand piece and via the galvo-controlled optical subsystem. The method may also include maintaining the galvo-controlled optical subsystem in a treatment position when the laser beam is ON and is directed to the designated dental area, and maintaining the galvo-controlled optical subsystem in a park position when the laser beam is OFF. In some embodiments, the method includes switching the galvo-controlled optical subsystem between the treatment position and park position to present an apparent persistent image to a viewer. For example, the switching can occur at least at 15 Hz.

### BRIEF DESCRIPTION OF THE DRAWINGS

The present invention will become more apparent in view of the attached drawings and accompanying detailed description. The embodiments depicted therein are provided by way of example, not by way of limitation, wherein like reference numerals generally refer to the same or similar elements. In different drawings, the same or similar elements may be referenced using different reference numerals. The drawings are not necessarily to scale, emphasis instead being placed upon illustrating aspects of the invention. In the drawings:

FIG. 1 depicts an overall laser-based system adapted for both treatment and imaging the area to be treated, according to one embodiment;

FIG. 2 illustrates a coupling between an imaging subsystem and an optical subsystem for directing a laser beam, according to one embodiment;

FIG. 3 illustrates another coupling between an imaging subsystem and an optical subsystem for directing a laser beam, according to one embodiment;

FIGS. 4-6 depict light sources for providing light to the treatment area, according to different embodiments; and

FIGS. 7 and 8 depict a coupling between imaging, illumination, and beam guidance subsystems, according to different embodiments.

### DETAILED DESCRIPTION

With reference to FIG. 1, a laser source can direct a laser beam into an articulating arm launch 1. The beam may be further directed within an articulating arm 2, and may exit therefrom at the end opposite the launch. In this laser-based dental treatment system, a main chamber 3 is connected to an interchangeable hand piece 4. One embodiment includes a variable speed foot pedal 6 to control the laser source and/or various other parameters of the dental system. A user interface (e.g., a touch screen input device) and/or monitor 5 can display images, and may be used to control various system parameters instead of or in addition to the foot pedal 6.

With reference to FIG. 2, in one embodiment, a main chamber 3 of a dental laser system houses an X Galvo 12 and a Y Galvo 13, and associated mirrors 14, 15 that are mounted on the X and Y galvanometers, respectively. The laser beam enters the module approximately along axis 20, reflects off the respective mirrors of X Galvo 12 and Y Galvo 13, is redirected through the hand piece 4 substantially along axis 17, reflects off a turning mirror 18, and exits the hand piece substantially along axis 19. In this embodiment, a camera assembly 6 including an image sensor 10, an optional filter 9, an optional fluidic lens 8, a lens stack 7, and an optional focusing motor 11 are mounted within the main chamber 3 such that the camera assembly can receive light reflected off the X Galvo mirror 14. Alternatively, or in addition, a camera assembly 16 can be mounted to receive light reflect off the Y Galvo reflective mirror 15. Both camera assemblies can receive light rays reflected from the dental treatment area, entering the handpiece along axis 19, reflecting off the turning mirror 18, propagating through the hand piece 4 along the optical axis 17, and reflecting off X Galvo and/or Y Galvo mirrors 14, 15, towards the image sensors in the respective camera assemblies.

The galvanometer mirrors 14, 15 may rotate into a “park” position that is not used during laser treatment, e.g., ablation. In the park position, the laser beam is typically switched off. When treatment is to be performed using the laser, the galvanometer mirrors may transition to a “treatment” position in which the mirror movement can be further controlled so as to deliver the laser beam to the treatment area, now turned on, according to a selected pattern. The camera assemblies 6, 16 are mounted such that the image capture can occur when the galvos 12, 13, and the associated mirrors 14, 15 are in the park position. When the laser beam is off, the operator can align the system by moving the hand piece so that the area to be treated is visible. Thereafter, the operator can switch to laser beam operation without moving the hand piece, or without having to interchange viewing, diagnostic, or treatment devices. The system may switch rapidly between “park” and “treatment” positions such that the operator can view the treatment area in near real time, as the area is being treated, without interrupting the laser treatment. This can be achieved if the rate of switching between park and treatment positions is at least 15 Hz, 25 Hz, 30 Hz, 50 Hz, and up to 100 Hz, or more.

FIG. 3 illustrates an embodiment of a main chamber 3 of a laser-based dental system that houses an X Galvo 12 with a transmissive optic 14 mounted thereon, and a Y Galvo 13 with a reflective mirror 15 mounted thereon. The laser beam can enter the main chamber 3 along axis 20, reflect off the transmissive optic 14 of the X Galvo 12 and the reflective mirror 15 of the Y Galvo 13. As such, the laser beam is redirected through the hand piece 4 substantially along axis 17. The laser beam may reflect off turning mirror 18 and may exit the hand piece 4 substantially along axis 19. A camera assembly 10 is mounted behind the transmissive optic 14, and can receive light rays reflected from the dental treatment area entering the hand piece 4 along the axis 19, reflecting off the turning mirror 18, propagating along the laser optical axis 17 through the hand piece 4, and through the transmissive optic 14. The transmissive optic 14 is substantially transparent to visible light but is reflective at the laser wavelengths, e.g., in a range from about 9 μm up to about 11 μm. In this embodiment, the source of illumination can be located as shown in FIG. 5.

As the camera assembly 10 receives the light reflected from the treatment area after such light passes through the transmissive galvanometer mirror 14, the camera assembly can capture the images of the area being treated even when the galvos 12, 13 and the associated optical components 14, 15 are in the treatment position. As such, switching rapidly between the park and treatment positions is not required, and the operator can view the treatment area in near real time as the area is being treated, without interrupting the laser treatment.

With reference to FIG. 4, in one embodiment, a main chamber 4 of a laser-based dental treatment system houses an X Galvo 12 and a Y Galvo 13 with reflective mirrors 14, 15 mounted on the two galvanometers 12, 13, respectively. The laser beam may enter the main chamber 3 along axis 20, reflect off the mirrors 14, 15, and be directed through the hand piece 4 along axis 17. The laser beam may then reflect off a turning mirror 18 and exit the hand piece 4 along axis 19.

A light source 21 is mounted generally perpendicularly to the axis 20 allowing the laser beam to pass through the light source 21. Light from the source 21 may also reflect off the reflective mirrors 14, 15, and may propagate through the hand piece 4 along the axis 17, reflect off the turning mirror 18, and may exit the hand piece along the axis 19. The light source 21 can include laser or light emitting diodes 22, 23. The diodes 22, 23 can be substantially single wavelength (or narrow spectrum) diodes, or multiple wavelength diodes. A Fresnel lens 24, or similar optically transmissive element, can be also be included in the light source, e.g., to minimize or eliminate stray radiation. As light from the illumination source 21 propagates through the main chamber 3 and the hand piece 4 substantially along the same path as that of the laser beam, the light from the source 21 can illuminate the treatment area during treatment. In this embodiment, the imaging sensors, i.e., cameras may be located as shown in FIG. 2.

An embodiment of a laser-based dental system shown in FIG. 5 is generally similar to that described with reference to FIG. 4. In this system, however, a light source 21 is mounted generally perpendicular to the axis 17, allowing the laser beam to pass through the light source 21. As such, light from the source 21 is directed through the hand piece 4 along the axis 17, may reflect off the turning mirror 18, and may exit the hand piece 4 substantially along the axis 19. The light source 21 can include narrow spectrum laser diodes or light emitting diodes and, optionally, a Fresnel lens, or similar optically transmissive element. As light from the illumination source 21 propagates through the hand piece 4 substantially along the same path as that of the laser beam, the light from the source 21 can illuminate the treatment area during treatment. In this embodiment, the imaging sensors can be located as shown in FIG. 2 and/or as shown in FIG. 3, if the optical element 15 is transmissive of visible light and reflects the laser beam.

Another embodiment of a laser-based dental system, depicted in FIG. 6, is similar to that described with reference to FIG. 5. In this embodiment, a transmissive optic 14 is mounted on the X Galvo 12, and a reflective mirror 15 is mounted on the Y Galvo 13. The light source 21 is located behind the transmissive optic 14, and can emit light therethrough, substantially along the axis 17. As such, light from the illumination source 21 can propagate through the hand piece 4 substantially along the same path as that of the laser beam, and can illuminate the treatment area during treatment. The transmissive optic 14 is substantially transparent to visible light but is reflective at the laser wavelengths, e.g., in a range from about 9 μm up to about 11 μm. In this embodiment, the image sensor can be located behind the transmissive optic 14 and/or behind the illumination source 21.

FIG. 7 depicts an embodiment of a main chamber 3 of a laser-based dental system that houses an X Galvo 12 and a Y Galvo 13 with reflective mirrors 14, 15 mounted on the two galvanometers, respectively. The laser beam may enter the main chamber 3 along the axis 20, reflect off the reflective mirrors 14, 15, and may be directed through the hand piece 4 along the axis 17. The laser beam may then reflect off the turning mirror 18 and exit the hand piece 4 along the axis 19.

A camera assembly that includes an image sensor 10, an optional filter 9, an optional a fluidic lens 8, a lens stack 7, and an optional focusing motor 11, is mounted so as to receive light reflected from the treatment area passing through or reflecting off a beam splitter 27. The beam splitter 27 can transmit the laser beam and, optionally, a marking laser, while redirecting the visible light reflected from the treatment area representing images thereof. Such light may enter the hand piece along the axis 19, reflect off the turning mirror 18, propagate along the optical axis 17, and may be reflected off the beam splitter 27 along the optical axis 28 towards the image sensor 10. An illumination system 33 can emit light from a light source 34 non-collinearly with the optical axis 17 via a lens 35. The illumination light may propagate through the hand piece 4, reflect off the turning mirror 18, and may propagate towards the treatment area with a waist along optical axis 19. The illumination light may be reflected from the treatment area, and may then be received by the image sensor 10, as described above.

FIG. 8 depicts an embodiment that is similar to the one described with reference to FIG. 7, except for the use of two beam splitters 27, 29 and for a different configuration of the illumination source. As in the embodiment described above with reference to FIG. 7, the image sensor 10 receives the light reflected from the treatment area after the light is redirected by the beam splitter 27, along the axis 28. That reflected light passes through another beam splitter 29, towards the image sensor 10. A light source 30 can emit illumination light along an optical path 31, which may reflect off the beam splitter 29 and propagate along an optical path 32. The illumination light may reflect again, off the beam splitter 27, and may then propagate towards the treatment area, similarly as described above with reference to FIG. 7. That light, upon reflection from the treatment area, can be received by the image sensor 10 after being reflected by the beam splitter 27 and passing through the beam splitter 29, as described above. In some embodiments, additional beam splitters can be added to provide illumination from different types of sources such as a broad-spectrum (e.g., from about 380 nm up to about 760 nm) white light source, narrow-spectrum red light source, a narrow-spectrum blue light source, etc.

In order to facilitate diagnosis via fluorescence or reflectance, the illumination source in some embodiments includes a predominantly monochromatic or narrow spectrum source of light, e.g., a laser diode or a light emitting diode (LED). In some embodiments, the illumination source emits red light in a range from about 600 nm up to about 700 nm. In other embodiments, the illumination source can emit blue light, e.g., in ranges from about 380 nm up to about 475 nm, from about 350 nm up to about 450 nm, from about 350 nm up to about 475 nm, or from about 375 nm up to about 475 nm. As this light is directed towards an area to be inspected, affected and unaffected areas may fluoresce differently and/or may reflect the light differently. The imaging system can capture these differences and allow the operator and/or another person to view the captured images to perform diagnosis based thereon. The images can be captured not only prior to treatment, but also when at least a part of the treatment is complete, but without requiring the operator to switch from a treatment device to a diagnostic/imaging device. Advantageously, the operator can assess the effectiveness of the partially completed treatment and/or evaluate a newly exposed portion of the tissue.

Various embodiments described above include one or more imaging devices. The imaging devices may include a CMOS or CCD chip coupled to a telephoto lens stack that can capture the image of the tooth on the imaging device. The lens stack may include a small motor, e.g., a squiggle motor, to move one or more elements of the lens stack so as to change the image size, focus, and/or to obtain an enlarged (e.g., zoomed in) or reduced (e.g., zoomed out) image. The motor typically includes a controller and an amplifier, and the motor control can be linked to the main system processor.

In some embodiments, to adjust the images, a liquid lens can be used. A voltage change can change the shape and/or size of the lens, thereby changing the image size and/or focus. The voltage can be adjusted to enlarge or reduce the image as well. Typically, the liquid lens is controlled by a voltage difference that can be adjusted by the main processor. Commands to the main processor for adjusting the lens stack motor position and/or pressure, size, and/or shape of the liquid lens, so as to adjust focus, size, and/or magnification of an image can be provided remotely, e.g., using a joystick, the foot pedal, touch screen controls, or other input devices.

In various embodiments, for illumination of the treatment area, white or broad-spectrum light from a corresponding source can be directed to such treatment area, via total internal reflectance in a light pipe or a light guide. The illumination source is typically adapted to ensure that substantially no stray white light is accidentally directed to any image sensor without first impinging upon and illuminating the tooth. In some embodiments, a marking or aiming laser beam is directed to the treatment area, typically collinearly with the treatment laser beam, to verify, for example, the spots/regions where the treatment laser beam would impinge when activated. The marking laser beam can be a monochromatic green laser beam having a wavelength of about 530 nm, and/or a monochromatic red laser beam having a wavelength of about 650 nm.

Using the user interface (UI) 5 (shown in FIG. 1), which can be a touch screen display, and/or other controllers, a wide array of hard and soft tissue procedures may be performed. By way of example and not of limitation, an operator may insert a hand piece into the patient's mouth and observe the image of the hard or soft tissue on the UI 5 or any other monitor. In various embodiments, the image displayed on the UI 5 may be captured by one or more image sensors, as described above. While viewing the tooth, the hand piece may be positioned to view the area of interest, e.g., an area to be treated. After positioning the hand piece in a selected position, the operator may activate the camera magnification using a controller, e.g., the foot pedal 6, the UI 5, etc. The operator may move or reposition the hand piece based on the changes in magnification.

The operator may also select a substantially monochromatic light (e.g., red or blue light), via a suitable input device such as the foot switch/pedal 6, the touch screen UI 5, etc. After the substantially monochromatic light is directed to the area of interest (e.g., a candidate treatment area), the operator may observe the different fluoresced color shades, and/or variations in the reflected light, on the user interface 5 or another monitor, to aid in the diagnosis of dental caries or any other condition in the candidate treatment area. The region of interest may be observed under different substantially monochromatic colors, e.g., red and blue.

Once the operator selects an area for treatment the operating parameters of the laser beam may be set, e.g., using the UI 5, the foot pedal 6, etc., and then the operator may activate the laser beam. While treating (e.g., ablating) hard or soft tissue, images of the tissue may be captured using the imaging system and be presented to the operator and/or other personnel in real time or in near real time, as described above. When the laser-based treatment procedure is at least partially complete the operator may turn the laser beam OFF and may inspect the tissue again. Diagnosis, via visual inspection/magnification, and/or by directing substantially monochromatic light and analyzing fluorescence and/or reflectance, can be performed again.

Such inspection and/or diagnosis during treatment can inform the remainder of the treatment, and the operator may adjust the treatment accordingly. For example, one or more laser beam parameters can be adjusted, the treatment area can be modified, and/or a new area may be identified for treatment. The operator may then reactivate the laser beam for further treatment, e.g., additional ablation, incision, etc. These alternating or interleaved steps of viewing, diagnosing, and treating can be repeated as often as determined to be necessary by the operator. Any combination of two or more steps can be performed in any order, without having to change instruments, and by holding a single hand piece in the patient's mouth.

While the invention has been particularly shown and described with reference to specific embodiments, it will be understood by those skilled in the art that various changes in form and detail may be made therein without departing from the spirit and scope of the invention as defined by the appended claims. The scope of the invention is thus indicated by the appended claims and all changes that come within the meaning and range of equivalency of the claims are therefore intended to be embraced.

What is claimed is:

1. An apparatus for imaging a dental treatment area, the apparatus comprising:
a galvo-controlled optical subsystem for directing a laser beam from a laser source to a dental treatment area, the laser beam being directed along an optical axis; and
an imaging system positioned at a location different than a location of the laser source, the imaging system comprising (i) a viewer, and (ii) an imaging optical subsystem adapted to receive light rays from the treatment area, the light rays traveling substantially along the optical axis and via the galvo-controlled optical subsystem, for delivery to the viewer.

2. The apparatus of claim 1, wherein the imaging optical subsystem comprises an adjustable focus lens mechanism to adjust focus of an image received in the viewer.

3. The apparatus of claim 2, wherein the adjustable focus lens mechanism comprises at least one of a motorized lens stack and a liquid lens.

4. The apparatus of claim 1, wherein the viewer comprises a camera.

5. The apparatus of claim 1, wherein the galvo-controlled optical subsystem comprises a first galvo-controlled mirror and a second galvo-controlled mirror.

6. The apparatus of claim 5, wherein the imaging optical subsystem is adapted to receive light rays reflected from one of the first and second galvo-controlled mirrors.

7. The apparatus of claim 5, wherein the first galvo-controlled mirror comprises an optically transmissive mirror, and the imaging optical subsystem is adapted to receive light rays passing through the optically transmissive mirror.

8. The apparatus of claim 1, further comprising an illumination system for directing light to the treatment area, the illumination system comprising a first light source.

9. The apparatus of claim 8, wherein the first light source comprises a light source of substantially monochromatic light.

10. The apparatus of claim 9, wherein the light source of substantially monochromatic light comprises at least one of a light emitting diode (LED) and a laser diode (LD).

11. The apparatus of claim 9, wherein the substantially monochromatic light has a peak wavelength range of one of about 600-700 nm and about 375-475 nm.

12. The apparatus of claim 8, wherein the first light source comprises an optically transmissive element.

13. The apparatus of claim 12, wherein the optically transmissive element comprises a Fresnel lens.

14. The apparatus of claim 8, wherein the first light source is located such that light therefrom is directed to the treatment area via the galvo-controlled optical subsystem.

15. The apparatus of claim 14, wherein the galvo-controlled optical subsystem comprises a galvo-controlled mirror, and the first light source is located such that light therefrom reflects off the galvo-controlled mirror.

16. The apparatus of claim 14, wherein the galvo-controlled optical subsystem comprises an optically transmissive element, and the first light source is located such that light therefrom passes through the optically transmissive element.

17. The apparatus of claim 8, wherein the first light source is located such that light therefrom is directed to the treatment area independently of the galvo-controlled optical subsystem.

18. The apparatus of claim 8, wherein the illumination system comprises a second light source.

19. An apparatus for imaging a dental treatment area, the apparatus comprising:
a first beam splitter;
a galvo-controlled optical subsystem for directing a laser beam from a laser source to a dental treatment area, the laser beam being directed via the first beam splitter, and along an optical axis;
an imaging system positioned at a location different than a location of the laser source, the imaging system comprising (i) a viewer, and (ii) an imaging optical subsystem adapted to receive light rays from the treatment area, the light rays traveling substantially along the optical axis and via the first beam splitter, for delivery to the viewer; and
an illumination system for directing light to the treatment area, the illumination system comprising a first light source of substantially monochromatic light.

20. The apparatus of claim 19, wherein the imaging optical subsystem comprises an adjustable focus lens mechanism to adjust focus of an image received in the viewer.

21. The apparatus of claim 20, wherein the adjustable focus lens mechanism comprises at least one of a motorized lens stack and a liquid lens.

22. The apparatus of claim 19, wherein the viewer comprises a camera.

23. The apparatus of claim 19, wherein the galvo-controlled optical subsystem comprises a first galvo-controlled mirror and a second galvo-controlled mirror.

24. The apparatus of claim 23, wherein the first galvo-controlled mirror comprises an optically transmissive mirror.

25. The apparatus of claim 19, wherein the first light source of substantially monochromatic light comprises at least one of a light emitting diode (LED) and a laser diode (LD).

26. The apparatus of claim 25, wherein the substantially monochromatic light has a peak wavelength range of one of about 600-700 nm and about 375-475 nm.

27. The apparatus of claim 19, wherein the first light source comprises an optically transmissive element.

28. The apparatus of claim 27, wherein the optically transmissive element comprises a Fresnel lens.

29. The apparatus of claim 19, wherein the first light source is located such that light therefrom is directed to the treatment area independently of the galvo-controlled optical subsystem.

30. The apparatus of claim 19, wherein the illumination system comprises a second light source.

31. The apparatus of claim 19, further comprising a second beam splitter wherein:
the laser beam is directed to the dental treatment area independently of the second beam splitter;
the imaging optical subsystem is located such that the light rays received thereby travel via the second splitter; and
the illumination system is located such that the light therefrom travels via the second splitter.

32. A method of identifying a dental area for laser treatment, the method comprising the steps of:
illuminating a candidate area for dental treatment using a source of substantially monochromatic light, the light passing via a hand piece adapted for passing therethrough and along an optical axis thereof a laser beam for treatment;
generating, at an imaging system, an image formed by light rays reflected from the candidate treatment area and traveling along the optical axis of the hand piece;
identifying within the generated image a region corresponding to received light rays having a wavelength different than a peak wavelength of the substantially monochromatic source of light; and
designating the identified region as the dental area for laser treatment.

33. The method of claim 32, further comprising:
directing a laser beam to the designated dental area via the hand piece, the laser beam being (i) generated by a source positioned at a location different than a location of the imaging system, and (ii) traveling substantially along the optical axis of the hand piece.

34. A method of treating a dental area using a laser, the method comprising the steps of:
directing a laser beam to a designated dental area via a galvo-controlled optical subsystem and via a hand piece along an optical axis thereof;
generating, at an imaging system, an image formed by light rays reflected from the designated dental area, the reflected light rays traveling along the optical axis of the hand piece and via the galvo-controlled optical subsystem.

35. The method of claim 34, further comprising:
maintaining the galvo-controlled optical subsystem in a treatment position when the laser beam is ON and is directed to the designated dental area; and
maintaining the galvo-controlled optical subsystem in a park position when the laser beam is OFF.

36. The method of claim 35, further comprising switching the galvo-controlled optical subsystem between the treatment and park positions to present an apparent persistent image to a viewer.

37. The method of claim 36, wherein the switching occurs at least at 15 Hz.
